(19) United States
(12) Patent Application Publication
(10) Pub. No.: US 2025/0319300 A1
(43) Pub. Date: Oct. 16, 2025

(54) INTRACAVITARY, PHYSIOLOGICAL AUGMENTATION DEVICE AND METHOD

(71) Applicant: Yale University, New Haven, CT (US)

(72) Inventors: Pramod Bonde, New Haven, CT (US); Rigu Gupta, New Haven, CT (US)

(21) Appl. No.: 19/175,271

(22) Filed: Apr. 10, 2025

Related U.S. Application Data
(60) Provisional application No. 63/632,249, filed on Apr. 10, 2024.

(51) Int. Cl. A61M 60/191; A61M 60/289; A61M 60/495; A61M 60/515; A61M 60/865

(52) U.S. Cl. CPC A61M 60/191; A61M 60/289; A61M 60/495; A61M 60/515; A61M 60/865; A61M 2230/04

(57) ABSTRACT

A physiological augmentation device for improving heart function includes an actuator configured to augment displacement of an atrioventricular plane of a heart. A method for improving heart function and a transcatheter method for improving heart function are also disclosed.

### CROSS-REFERENCE TO RELATED APPLICATIONS

This application claims priority to U.S. provisional application No. 63/632,249, filed Apr. 10, 2024 incorporated herein by reference in its entirety.

### BACKGROUND OF THE INVENTION

Understanding cardiac pumping function is crucial to improving diagnosis, predicting the outcomes of intervention, and designing medical devices that interact with the cardiovascular system. The pumping physiology of the heart has been differently described over the years [1], [2], [3]. A common approach is to explain the filling and emptying of the heart in terms of a radial squeezing of the cardiac chambers [4], [5] with considerable changes in total heart volume throughout the cardiac cycle, as supported by observations conducted during open heart surgery. However, constraints induced by the pericardium and the tissues surrounding the heart in situ strongly limit these volume changes [6], [7]. More specifically, the myocardium can slide along the pericardium owing to a thin layer of pericardial fluid, but it cannot detach from it or stretch the pericardium extensively. In this way, any unnecessary displacement of the surrounding tissues and consequent energy loss are avoided. However, this energy-efficient pumping means that the reduction in ventricular volume during the ejection of blood into the arteries has to be compensated for by some internal redistribution of blood within the cardiac chambers.

The reciprocal blood redistribution between atria and ventricles is achieved by internal longitudinal movements of the atrioventricular (AV) plane, of which initial measurements were reported in the pioneer work from Hoffman and Ritman [8]. The atrioventricular plane is a fibrous region including the cardiac valves and separating the atria from the ventricles. The relative constancy of the cardiac volume throughout the cardiac cycle as well as reciprocal volume changes have also been shown in other studies [9], [10]. Based on the aforementioned considerations, it has been suggested that the heart can be described as a displacement pump in which the AV plane behaves as a piston unit [11], [12], [13]. This dome-shaped piston unit is composed of the rings surrounding all four cardiac valves (the fibrous skeleton of the heart) and the muscles attached to this semi-rigid anatomical structure.

Throughout the cardiac cycle, the AV plane moves back and forth in the base-apex direction, creating reciprocal volume changes between atria and ventricles while keeping an almost constant total heart volume. The AV-plane moves as a piston pump with each heartbeat, towards the apex during systole then back to the original position during diastole. During ventricular systole, the AV plane is pulled towards the apex by the shortening of cardiac muscle, generating a volume displacement in the direction of the arteries (the stroke volume of the heart). At the same time, this downward movement produces atrial expansion, allowing for inflow from the veins to the atria, which results in the quantitatively most important contribution to atrial filling [14]. During ventricular diastole, the AV plane moves back to its initial position, redistributing blood from the atria to the ventricles (the ventricular filling volume).

These longitudinal movements can be clearly observed using different imaging modalities, proving that this way of pumping differs from the traditionally accepted squeezing mechanism. The left ventricular ejection pattern was vividly described by Rushmer as more similar to “striking a piston with a mallet than squeezing an orange or milking a cow” [15]. More recently, this theory has been quantitatively supported by cardiac magnetic resonance imaging (MRI) in healthy individuals that shows the distinct movement of the AV plane with approximately fixed apex and minor changes in total heart volume [3], [10]. Furthermore, the clinical value of measuring AV-plane displacement has been proved in various studies [16], and the magnitude of the AV-plane displacement has been shown to be a reliable index for diagnosing heart failure owing to the strong correlation of AV-plane displacement, ejection fraction, and stroke volume [3], [18].

Meanwhile, heart pumps that support blood circulation have several drawbacks, including bleeding due to blood damage; thrombosis and pump clots due to blood damage and shear and stress; massive power needs; the need for obligatory continuous operation lest backward flow from ventricle to aorta will cause catastrophic heart failure; they work in parallel and compete with the hearts physiological function; and infection at driveline and pump pocket due to penetrating wires needed to supply huge amounts of power.

Accordingly, there is a need in the art for improved devices and methods for treating heart dysfunction that do not have the drawbacks of conventional heart pumps while taking advantage of the strong correlation between AV-plane displacement, ejection fraction, and stroke volume. The embodiments described herein fit this need.

### SUMMARY OF THE INVENTION

A physiological augmentation device for improving heart function includes an actuator configured to augment displacement of an atrioventricular plane of a heart. In one embodiment, the displacement comprises movement along an axis substantially perpendicular to the atrioventricular plane. In one embodiment, the actuator is configured to be coupled to the heart. In one embodiment, the actuator is configured to be coupled to a top portion of the heart. In one embodiment, the actuator comprises a fibrous ring configured to be coupled to the heart. In one embodiment, the actuator is configured to be disposed substantially along the axis. In one embodiment, the actuator is configured to augment displacement of the atrioventricular plane between 1 millimeter and 7 millimeters. In one embodiment, the actuator is configured to augment displacement of the atrioventricular plane between 3 millimeters and 7 millimeters. In one embodiment, the actuator is configured to augment displacement of the atrioventricular plane between 5 millimeters and 7 millimeters. In one embodiment, the actuator is configured to augment displacement of the atrioventricular plane by about 7 millimeters. In one embodiment, the actuator comprises a plurality of magnets. In one embodiment, the plurality of magnets comprises a first magnet construct and a second opposing magnet construct configured to convert magnetic energy into linear motion along the axis. In one embodiment, the first magnet construct is configured to energize based on a measured electric signal from the heart. In one embodiment, the first magnet construct comprises at least one sliding magnet. In one embodiment, the actuator comprises a rotary device configured to convert rotary motion into linear motion along the axis. In one embodiment, a second actuator is configured to change a ring geometry within the atrioventricular plane. In one embodiment, a secondary element is configured to augment movement of a papillary muscle for generating rotational movement.

In one embodiment, a method for improving heart function includes the step of augmenting displacement of an atrioventricular plane of a heart. In one embodiment, the displacement comprises movement along an axis substantially perpendicular to the atrioventricular plane. In one embodiment, the displacement is initiated by an actuator. In one embodiment, the actuator is coupled to the heart. In one embodiment, the actuator is coupled to a top portion of the heart. In one embodiment, the actuator is coupled to the heart via a fibrous ring. In one embodiment, the actuator is disposed substantially along the axis. In one embodiment, the method includes augmenting displacement of the atrioventricular plane between 1 millimeter and 7 millimeters. In one embodiment, the method includes augmenting displacement of the atrioventricular plane between 3 millimeters and 7 millimeters. In one embodiment, the method includes augmenting displacement of the atrioventricular plane between 5 millimeters and 7 millimeters. In one embodiment, the method includes augmenting displacement of the atrioventricular plane by about 7 millimeters. In one embodiment, the method includes changing a ring geometry within the atrioventricular plane. In one embodiment, the method includes augmenting movement of a papillary muscle for generating rotational movement. In one embodiment, the method includes augmenting movement of both the left and right ventricles.

In one embodiment, a transcatheter method for improving heart function includes the steps of advancing a catheter though a vascular system towards a heart; deploying an actuator device from a distal opening of the catheter; retracting the catheter through the vascular system; and augmenting displacement of the atrioventricular plane of the heart via the deployed actuator device. In one embodiment, the displacement comprises movement along an axis substantially perpendicular to the atrioventricular plane. In one embodiment, the actuator is coupled to the heart. In one embodiment, actuator is coupled to a top portion of the heart. In one embodiment, the actuator is coupled to the heart via a fibrous ring. In one embodiment, the actuator is disposed substantially along the axis. In one embodiment, the method includes augmenting displacement of the atrioventricular plane between 1 millimeter and 7 millimeters. In one embodiment, the method includes augmenting displacement of the atrioventricular plane between 3 millimeters and 7 millimeters. In one embodiment, the method includes augmenting displacement of the atrioventricular plane between 5 millimeters and 7 millimeters. In one embodiment, the method includes augmenting displacement of the atrioventricular plane by about 7 millimeters. In one embodiment, the method includes changing a ring geometry within the atrioventricular plane. In one embodiment, the method includes augmenting movement of a papillary muscle for generating rotational movement. In one embodiment, the method includes augmenting movement of both the left and right ventricles.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A shows a diagram of atrioventricular plane displacement (AVPD). FIG. 1B shows a functional view of an augmentation device according to one embodiment; FIG. 1C shows a functional view of an augmentation device with suction applied during implantation according to one embodiment, FIG. 1D shows a functional view of an augmentation device with a magnetic fixation construct for target positioning according to one embodiment; and FIG. 1E shows a functional view of an augmentation device with in-plane adjustment mechanisms according to one embodiment.

FIGS. 2A and 2B show alternate mechanisms for producing linear movement, including a sliding magnet (FIG. 2A) according to one embodiment and a rotary cam construct (FIG. 2B) according to one embodiment.

FIGS. 3A-4Q show various functional views according to various embodiments.

FIGS. 5A-5D show an experimental example of a device and method for improving heart function, including a device having an annuloplasty ring (FIGS. 5A and 5B) and a device having a webbed conical structure (FIGS. 5C and 5D). FIGS. 5E and 5F are tables showing experimental results.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods for improving heart function. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a physiological augmentation device and method for improving heart function.

Embodiments of the augmentation device and method described herein are inserted by a catheter and sit within the heart, augmenting blood flow and output from the heart by exploiting the physiological processes of its function. Embodiments include a pumpless design without need for impellers, without stress or shear of blood and without need for long term anticoagulation. Embodiments of the device and method works in concert with the heart's pumping action rather than against or in parallel to it.

Devices according to embodiments described herein may utilize a catheter delivered AVPD plane augmentation ring anchored to a mitral valve. Movement of the device gated with EKG augments the filling and emptying of the left ventricle and increases stroke volume (blood ejected per beat) by for example 60% and cardiac output (blood ejected per minute) by for example 30%. The atrial septum can be sandwiched between a wired construct allowing side to side movement that increases atrial filling and emptying contributing further for example 20% of the stroke volume. The movement can be achieved by for example converting rotary motion to linear motion or by using linear electromagnets. Other examples may include for example small scale mechanical systems, rotary to linear conversion via threaded surfaces (e.g. a threaded shaft that rotates within a threaded nut, or a similar ball screw construct); a rack and pinion system; a pneumatic or hydraulic system using fluid power to generate linear motion by applying pressure to a piston within a cylinder; a linear motor producing linear motion without the need for conversion from rotary motion; a linear actuator such as a solenoid, piezoelectric actuator or mechanical linkage; or electro-mechanical systems such as stepper motors and linear encoders.

In one embodiment, a battery and circuitry are implanted similar to a pacemaker below the collar bone and the battery has 24 hours run time. The battery is charged using wireless power transfer. Non-functioning due to power depletion does not affect the normal functioning of the ventricle/atria and does not pose a challenge and the device simply switches on once powered. Embodiments of the device can be used in various configurations and even during arrythmias.

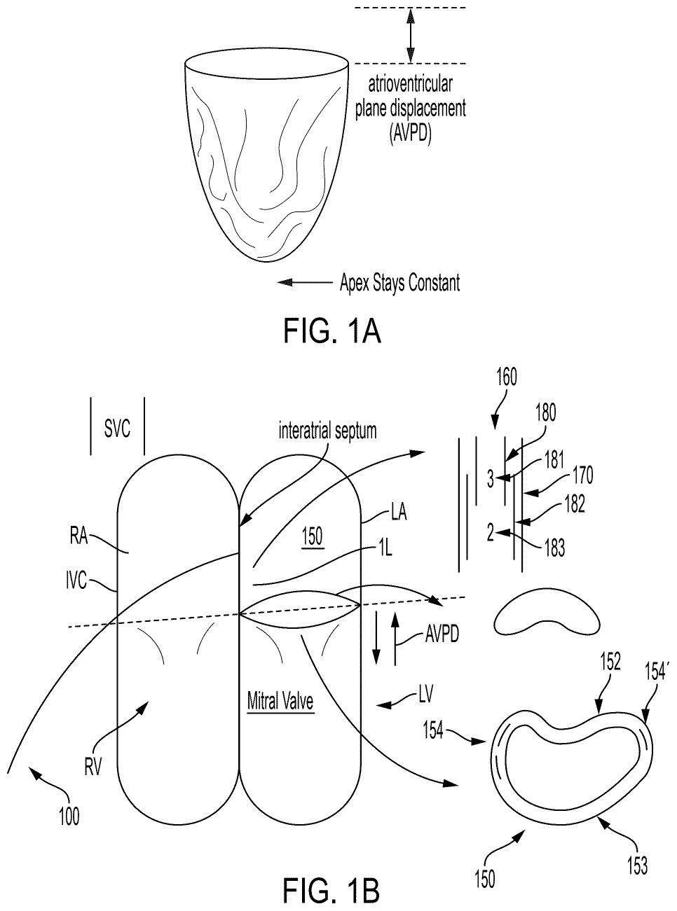

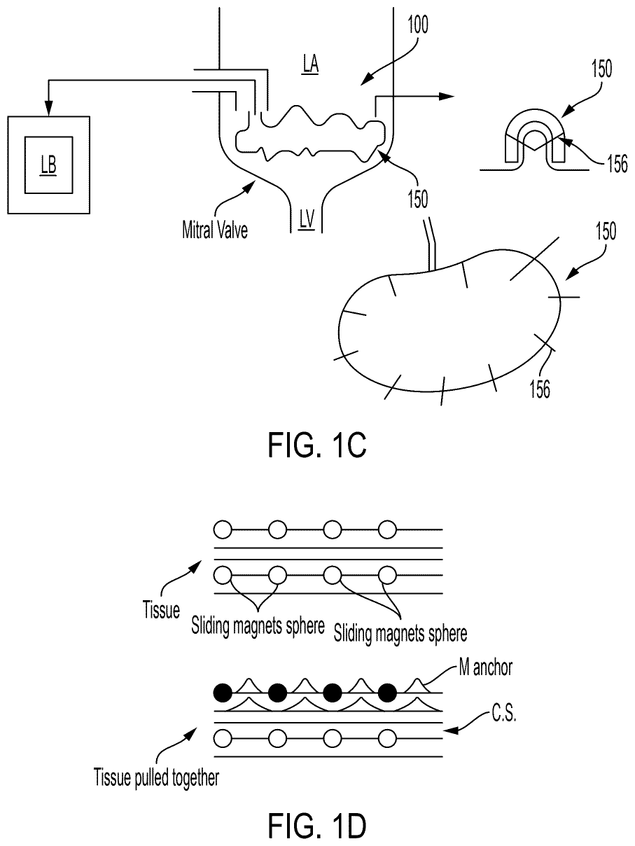

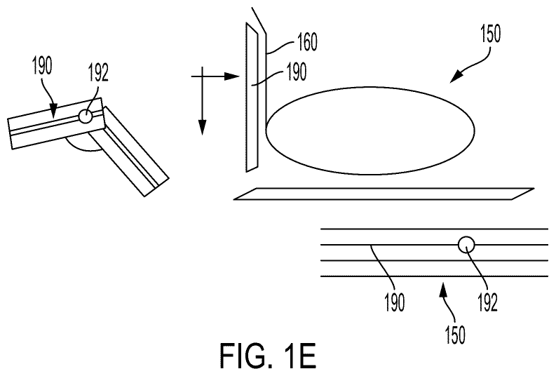

Advantages of embodiments of the device and method will now be explained in more detail with reference to the heart's anatomy and function. The heart is contained within a cavity called the pericardium. The pericardium is a sac that contains only about 15 to 20 CC's of fluid in it. Increasing fluid in the pericardial sac, even by only for example 50 CCs, will impair functioning of the heart. The pericardial sac is also non-compliant, meaning the size of the pericardial sac will not stretch or otherwise increase. Studies of heart images have shown that the apex of the heart remains substantially stationary during pumping (e.g. FIG. 1A). While echocardiograms only show one section of the heart, the improvement of cardiac MRI technology has been able to demonstrate that movement of the heart is in fact an up and down sliding movement through the cardiac cycle within the pericardial sac along the atrioventricular plane. Thus, the relationship between the upper chambers and the lower chambers is essentially that of a positive displacement piston pump. The left and right chambers slide up and down within the pericardial sac along the atrioventricular plane acting as a positive displacement pump through the cardiac cycle. The amount of atrioventricular plane displacement may typically range upwards of 15 to 20 mm of movement.

In a normal individual, the left and right chambers move back and forth along an axis perpendicular to the atrioventricular plane. As the atrioventricular plane moves, chambers of the heart fill and empty. Not much difference is observed in side-to-side motion. “Squeezing” of the heart during the cardiac cycle is actually a misconception, since as explained above the movement is actually a piston-like movement that goes up and down referred to as atrioventricular plane displacement or AVPD. As the pericardium surrounds the heart and is non-compliant, the heart cannot suddenly collapse or squeeze. So the heart instead employs the piston-like positive displacement pumping motion, moving back and forth along the atrioventricular plane. Displacement of the atrioventricular plane (AVP) is responsible for approximately 60% contribution to stroke volume and 30% of ejection fraction.

Studies have measured AVPD is groups of people including a healthy normal control group, athletes and people with heart failure. In one study, the healthy normal control group measured about 17 mm, athletes about 17.5 or 18 mm, however people with heart failure are reduced to only about 7 mm or so. Stroke volume is the amount of blood that the heart ejects with every beat, normally about 70 CCs to 120 CCs. The controls ejected about 110 CCs per contraction, the athletes ejected about 135 CCs per contraction, while the patients with heart failure are reduced to only about 70 CCs or so, almost half of what would normally be expected. Atrioventricular plane displacement contributes 60% of the stroke volume, or in other words 60% of the stroke volume comes from the movement of the atrioventricular plane. That in turn becomes 30% of the ejection fraction (the fraction or ratio of the blood before then after the heart ejects).

Thus in patients with heart failure, movement of the atrioventricular plane is greatly reduced. This has been confirmed by numerous echocardiographic and MRI studies. Atrioventricular plane movement has been demonstrated to correlate with the severity of heart disease. The degree of the NYHA correlates to the degree of heart failure, (I) being the list severe, and (IV) being the most severe. As the atrioventricular plane displacement decreases, there is more severe heart failure. The same strong correlation between atrioventricular plane displacement and the degree of the heart failure.

Data shows that atrioventricular plane displacement also correlates with survival of the patient. Data demonstrates that if the atrioventricular plane moves by 8.8 mm or more, chances of surviving to about five years or so are almost all 90% (a good survival rate). Once it drops to between 8.8 to 6.8 mm, survival will be up to 90% for maybe up to two years. However, by the time you reach five years, it is as little as about 85% or so. If the plane movement is less than 6.8 mm by the end of five years the survival rate is only about 75%. The data shows a very solid correlation between atrioventricular plane displacement and survival rates.

With reference now to FIGS. 1B-1E, a physiological augmentation device for improving heart function is shown according to one embodiment. With reference now to FIG. 1B, a functional view of a device 100 according to one embodiment is shown. FIG. 1B depicts elements including sheath and cathter via IVC (100), mitral annulus (near), posterior, area adjacent to annulus (150), mitral annulus, anterior (152), annulus proper (153), junction of anterior and posterior annulus (also called commissure) (154), inner sheath lumen (160), outer sheath (170), inner sheath/catheter (180), middle sheath, catheter in telescoping arrangement (182) and lumen of middle catheter/sheath (183). The device 100 is shown advanced and deployed into chambers of the heart. The device 100 includes a semi-rigid ring 150 that in at least one portion includes a shape memory flexible portion 152 that can depress and elevate. The elevation can be passive such that in a relaxed state, the shape memory flexible portion 152 maintains an elevated position. The relaxed state elevated position can be designed to match the desired amount of augmented atrioventricular plane displacement (e.g. 7 millimeters of augmented displacement). The semi-rigid ring 150 may include a set of pivot points 154, 154′ that separate the shape memory flexible portion 152 form an opposing portion 153 that may also be collapsable and made from a shape memory material, but otherwise has a different property such as added rigidity so that it does not flex upon depression or elevation of the shape memory flexible portion 152.

The device is designed to collapse into a delivery catheter 170, then deploy from a distal opening of the delivery catheter 170 once the delivery catheter is advanced to the target site within the left atrium. Embodiments can gain access from a femoral vein to the right atrium from the inferior vena cava (IVC) or the superior vena cava (SVC). A transseptal puncture needle can be advanced to the interatrial septum, allowing the delivery catheter 170 to then cross the interatrial septum so that the device 100 is ready to deploy into the left atrium. Loading, advancement and deployment can be similar to stent implantation and the ring may include stent constructs to from the desired shape and structural or functional characteristics. Embodiments of the device 100 may include coaxially loaded sliding portions 180, 182 that are telescoped for creating linear motion and generating atrioventricular plane displacement. The telescoped sliding portions 180, 182 include a series of electromagnets 181, 183 that can activate for generating linear movement of the telescoping arm 160. The telescoping arm 160 not only depresses but can also manipulate the posterior part of the semirigid ring 150. The telescoping arm 160 and telescoped sliding portions 180, 182 may be housed inside of an external device shaft. The arm 160 if offset instead of centered to avoid obstructing the mitral valve.

With reference now to FIG. 1C, embodiments of the device 100 can connect at a proximal end to a suction mechanism that generates suction at the semi-rigid ring 150 for implantation. The semi-rigid ring 150 includes barbs or anchors 156 positioned along portions of the annulus that are configured to suction-up tissue, allowing the barbs or anchors to fire and set the semi-rigid ring 150 into place atop the mitral valve. Multiple anchoring points are positioned around the semi-rigid ring 150 for stability. Once the anchors are engaged, the delivery catheter 170 can be retracted. A magnetic guide wire can be used in conjunction with a second magnetic guide wire construct near the aortic valve to confirm correct positioning prior to deployment of anchors (see e.g. FIG. 1D, showing an application of a magnetic construct allowing one to be placed within the coronary sinus and other within the left atrium to cinch the tissue and allow actuation according to one embodiment). The second opposing electromagnet construct can be positioned to capture the ring and stick it to correct position from below, then once fixated in position, suction can be activated capture tissue and set the anchors in place.

Embodiments of the device include a controller connected to electromagnetic constructs 181, 183 and may also be connected to physiologic sensors such as sensors for detecting heart rhythms for pacing actuation. The controller can be implanted like a pacemaker box that includes a battery and control mechanisms. The shaft driven mechanism described above activates via the controller by activating electromagnets to produce linear motion. In one embodiment, with reference now to FIG. 1E, the ring 150 can be broken into multiple section (e.g. 12 sections) and all those sections each have a wire that can be manipulated through the arm 160 for manipulating movement of the ring. Each section can be connected by a pivot point 192 to create a lever mechanism which allows each section to move at least partially or fully independent of other sections. This allows for additional movement and distortion capabilities for customizing ring position, shape and movement, and manipulating ring diameter. Accordingly, not only can embodiments of the device move up and down for augmenting atrioventricular plane displacement, they can also manipulate movement within the atrioventricular plane itself, allowing for three dimensions of mechanical augmentation.

A physiological sensor such as an EKG sensor can be detected to trigger actuation. Peak amplitude is detected for gating the pacing signal. Advantageously, embodiments of the device unlike a conventional pump do not have to run continuously. The device only need to activate for a fraction of the cardiac cycle. So for example, if a person's heart is beating at 70 beats per minute, out of the 0.8 seconds it takes about 0.5 seconds for the chamber to fill and 0.3 seconds for it to eject. So the device only need to active and augment for about 0.3 seconds to actively depress, and for the remaining 0.5 seconds it's passively elevating itself. The device is also working with the heart, not against the heart. So the amount of force needed to apply depression is very small, and power consumption is also minimal allowing the device battery to last a long time and providing a more durable and predictable device functionality.

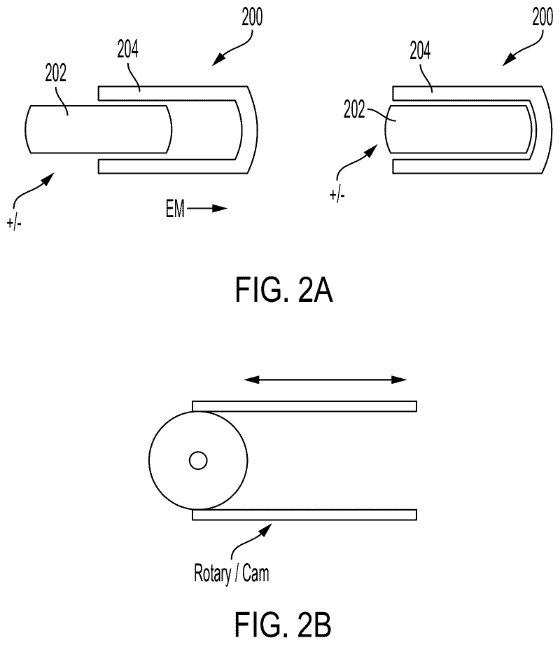

With reference now to FIGS. 2A and 2B, alternate mechanisms for producing linear movement in the device are show including electromagnetic sliding and capture to move the annular plane up and down (top) and a rotary/cam arrangement for achieving up and down motion (bottom). As shown in FIG. 2A, a sliding magnet 200 includes an internal magnet 202 partially loaded into a lumen of an external sleeve 204. The internal magnet 202 can be energized to and move linearly within the external sleeve 204 (or vice versa) for producing a linear motion. One or more sliding magnets can be configured into the device arm for actuating the ring. As shown in FIG. 2B, a rotary cam construct an alternatively be implemented to turn rotary motion into linear motion. One or a combination of these types of constructs can be incorporated into the device for actuation.

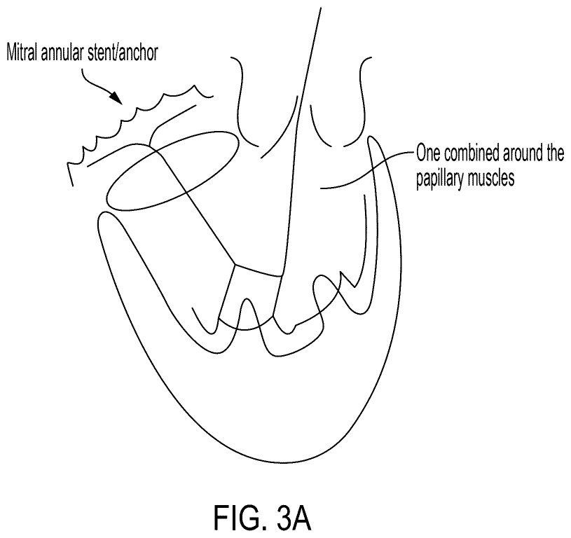

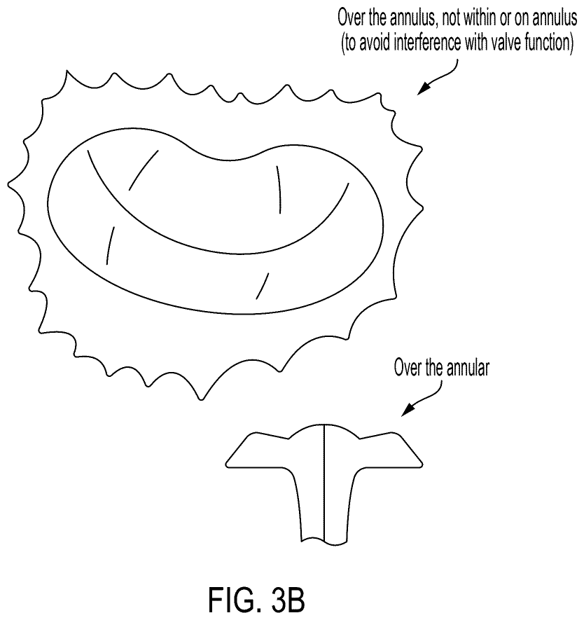

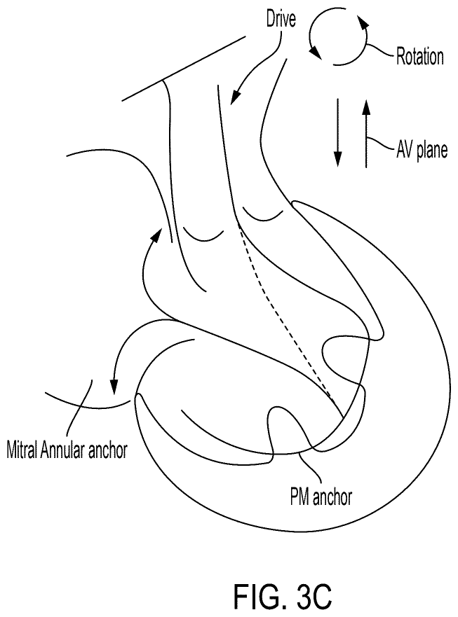

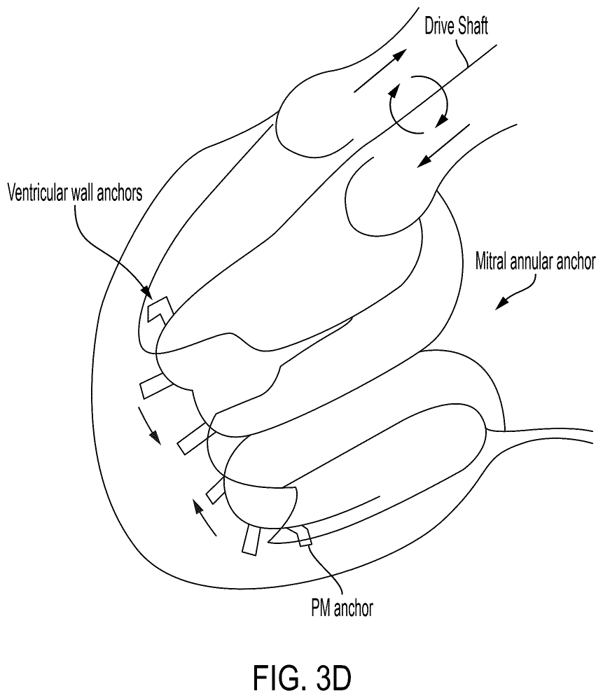

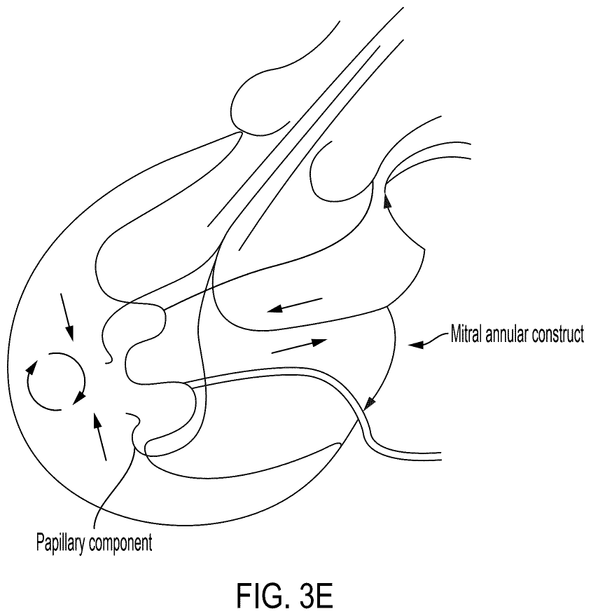

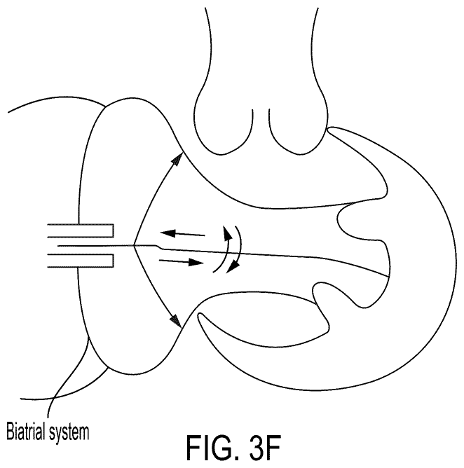

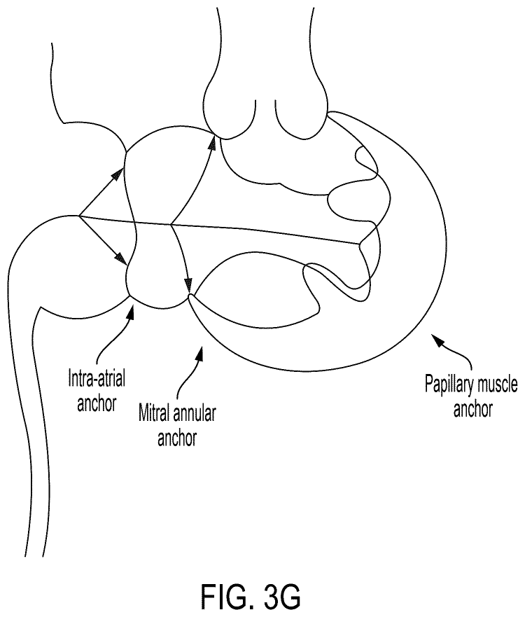

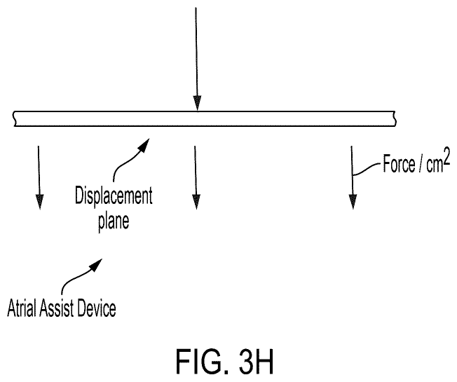

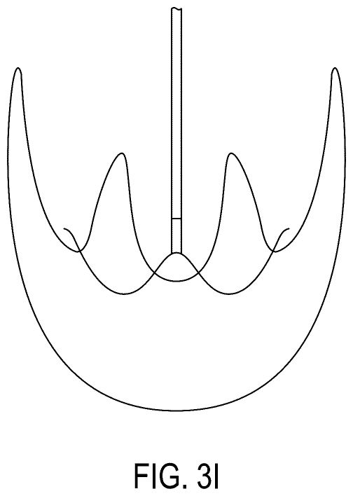

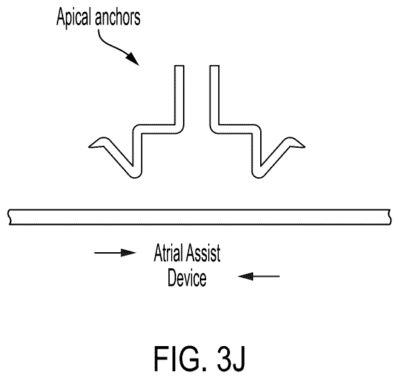

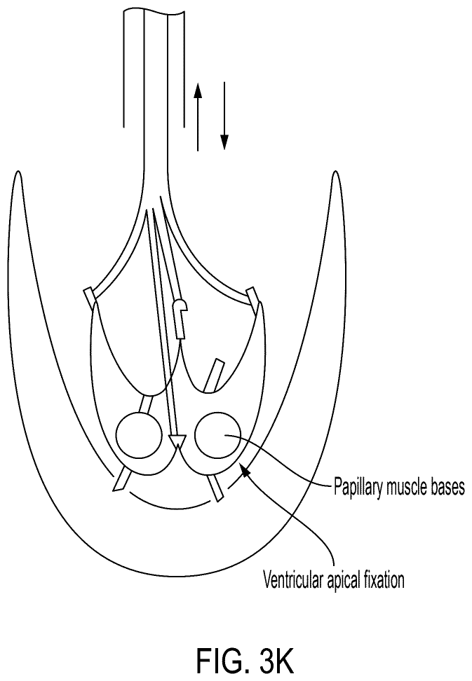

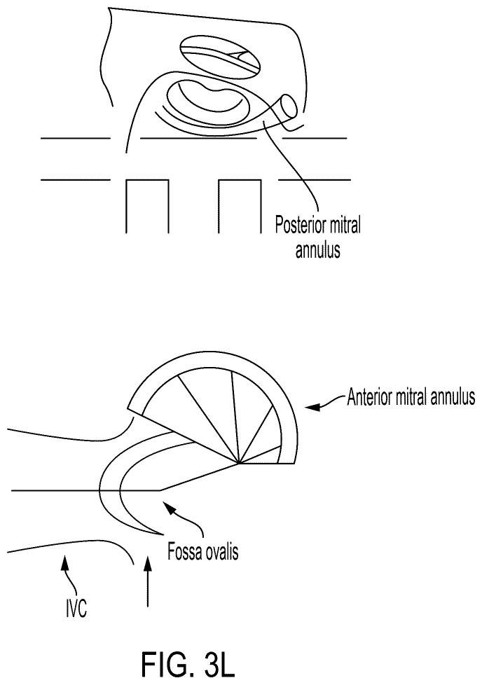

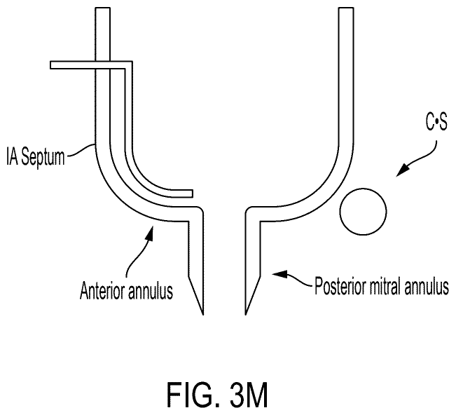

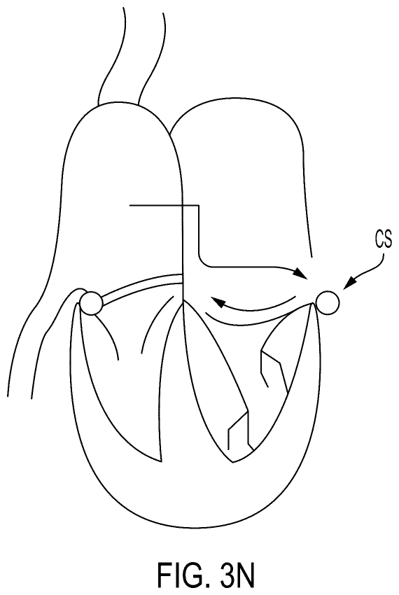

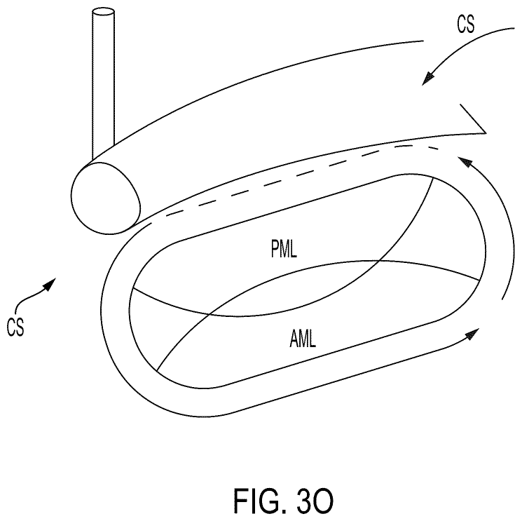

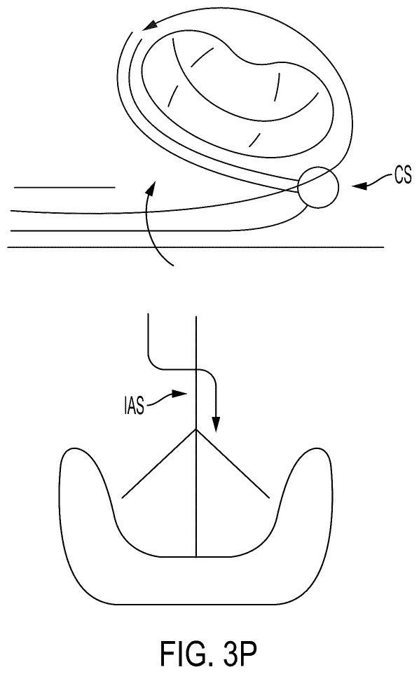

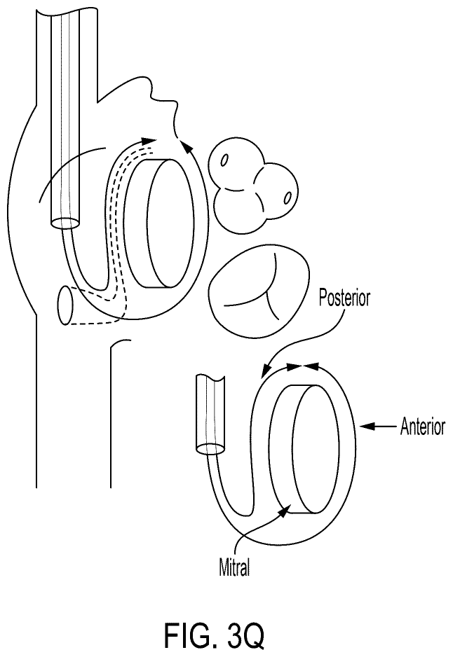

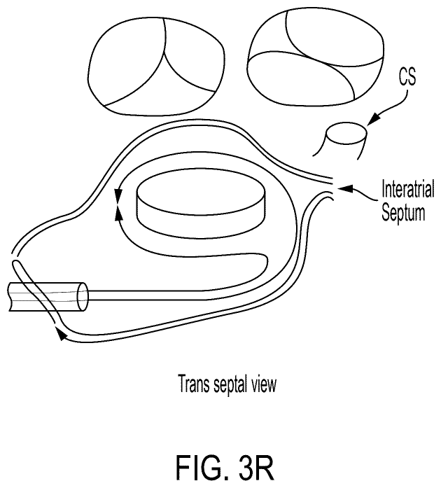

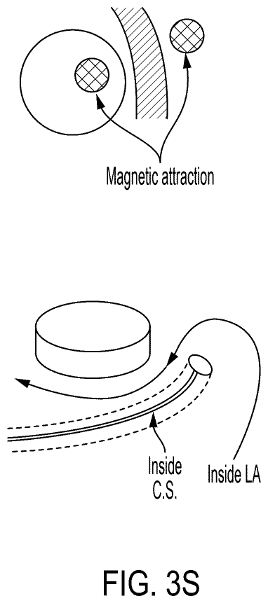

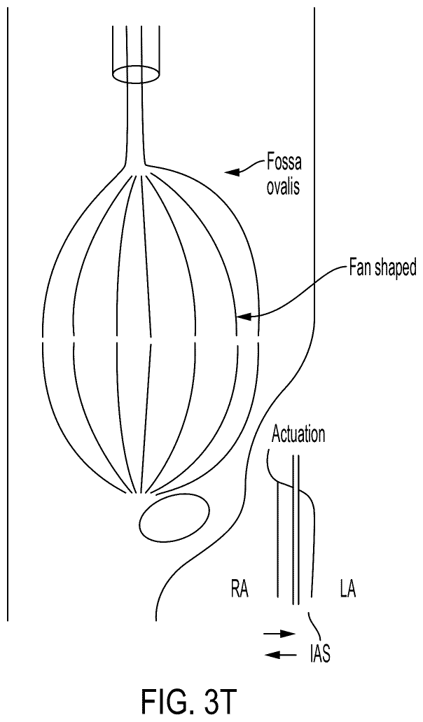

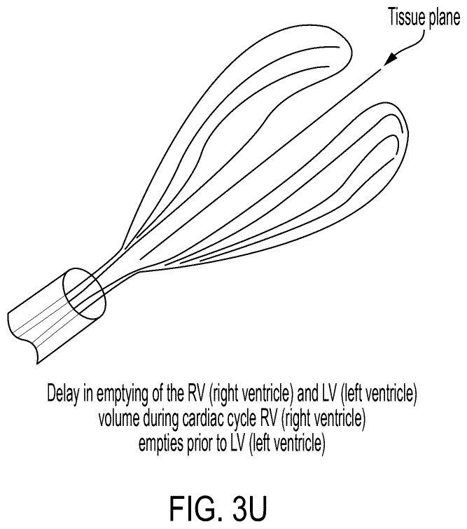

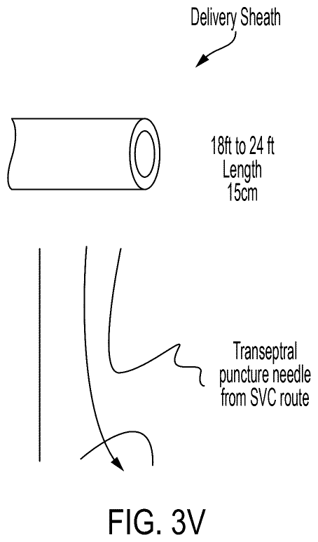

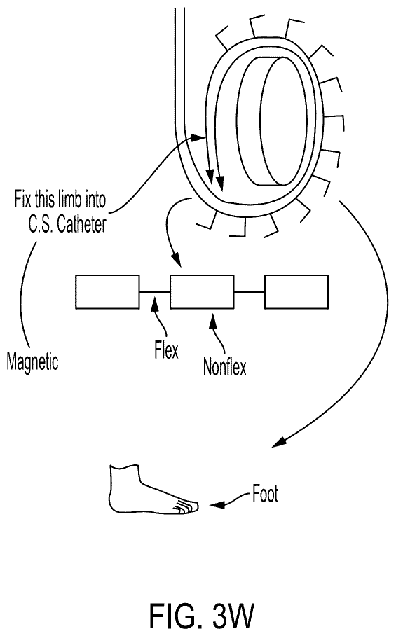

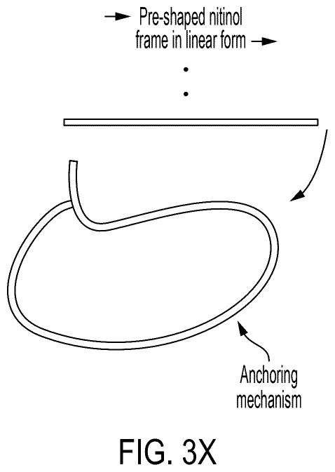

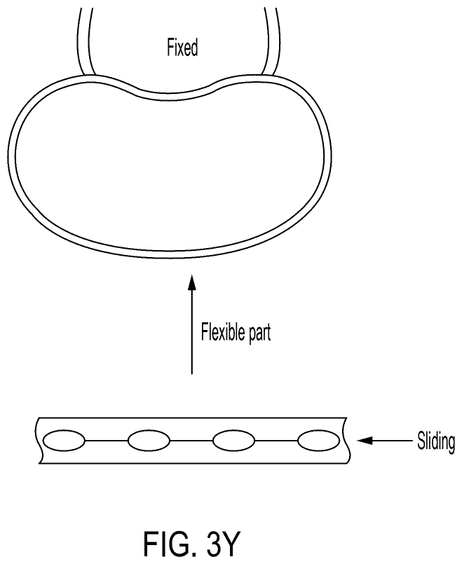

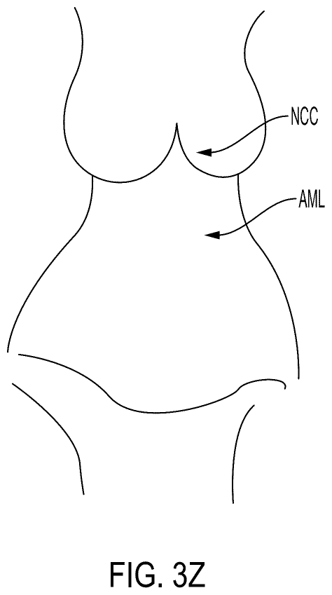

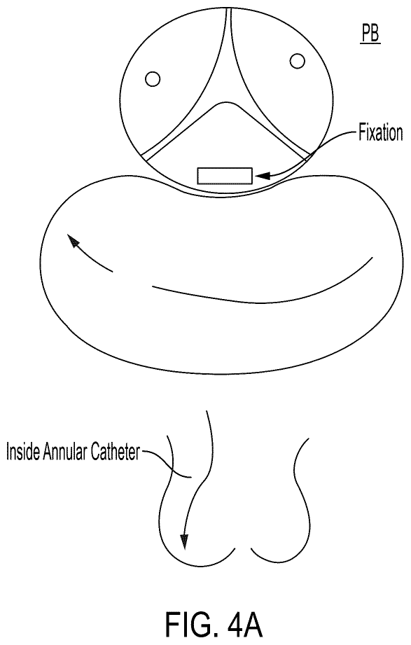

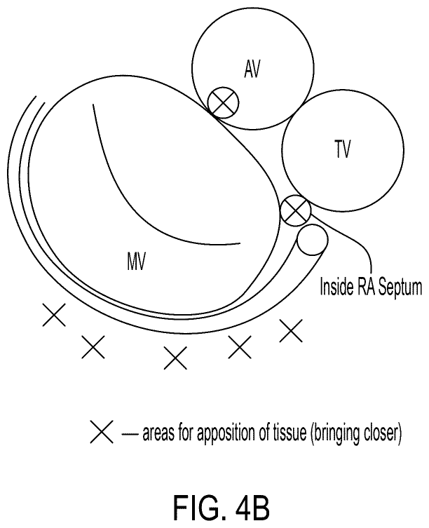

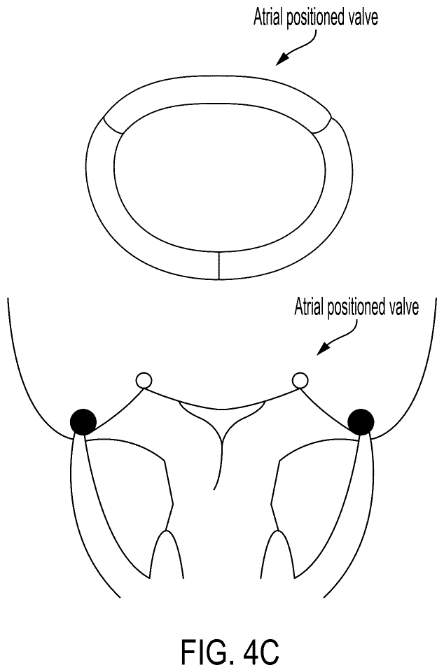

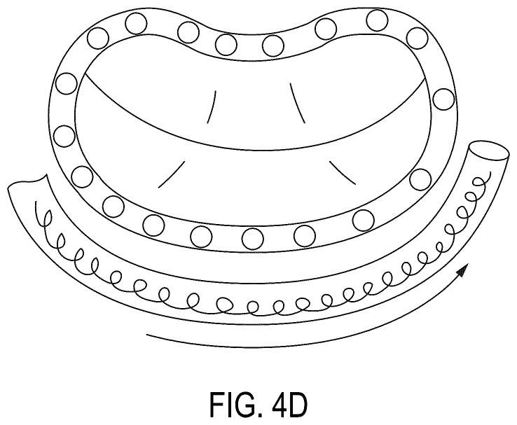

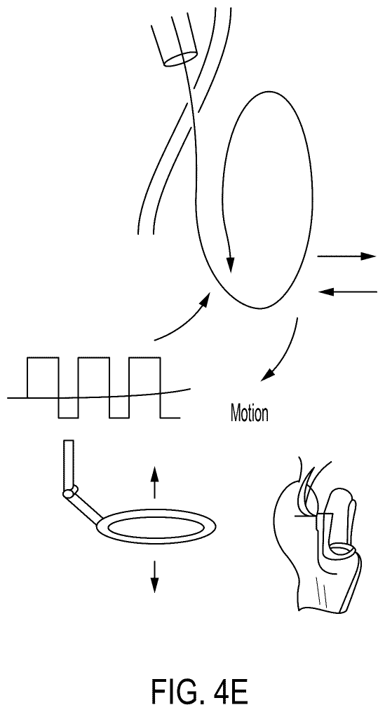

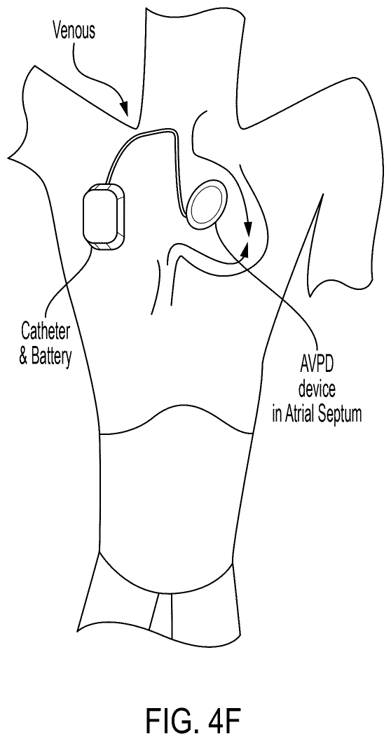

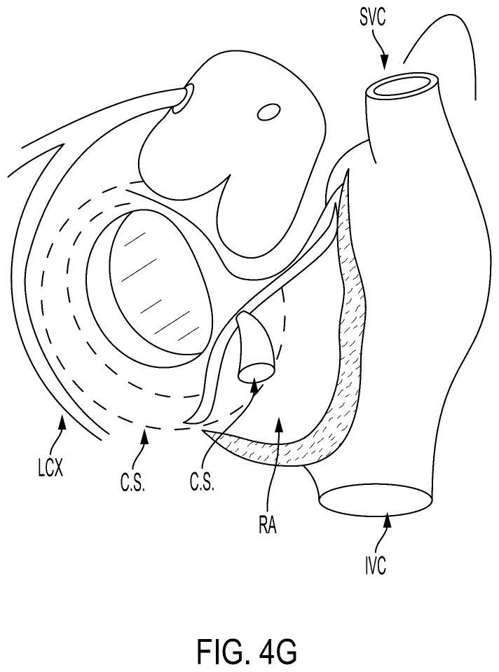

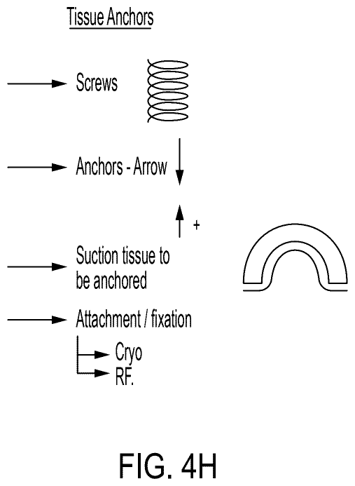

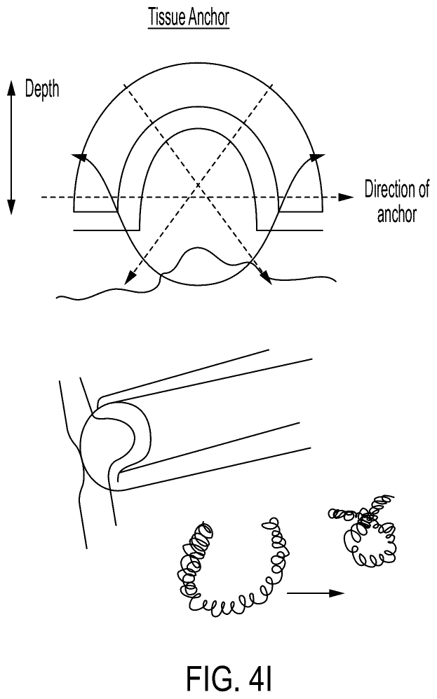

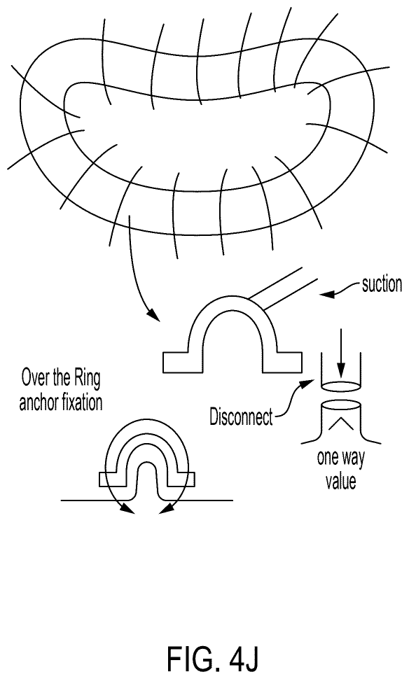

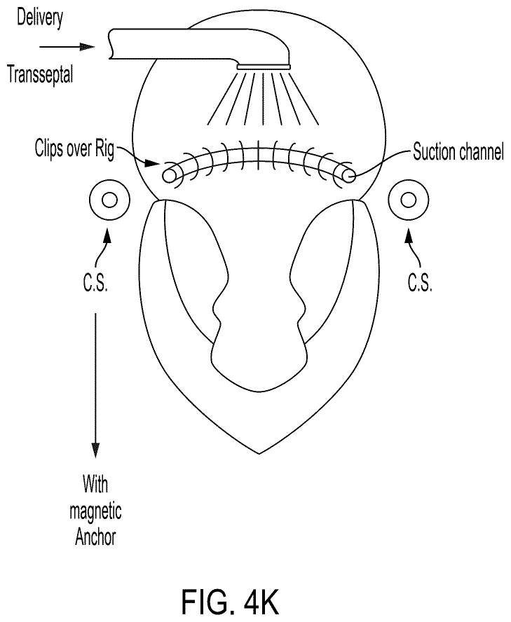

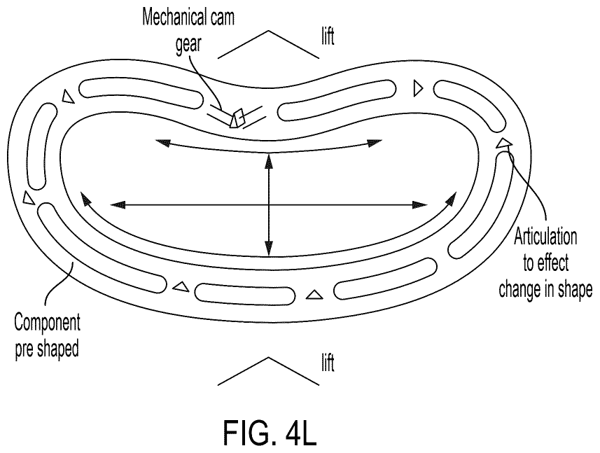

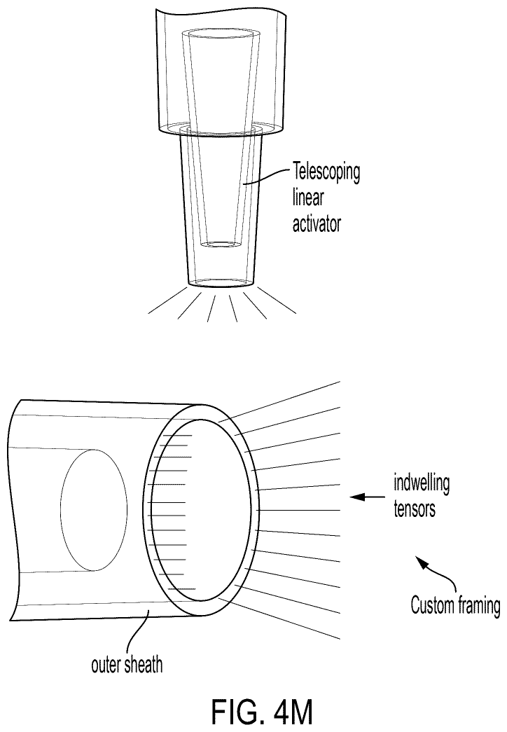

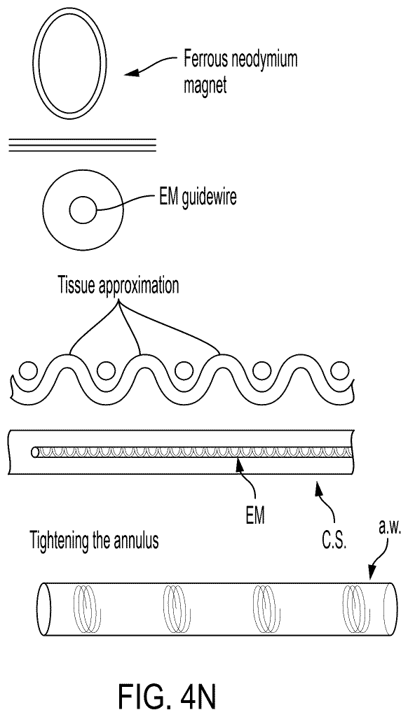

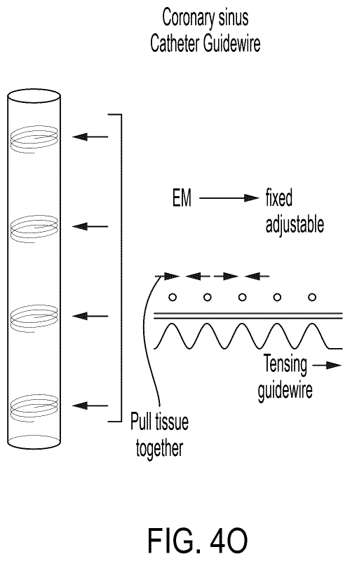

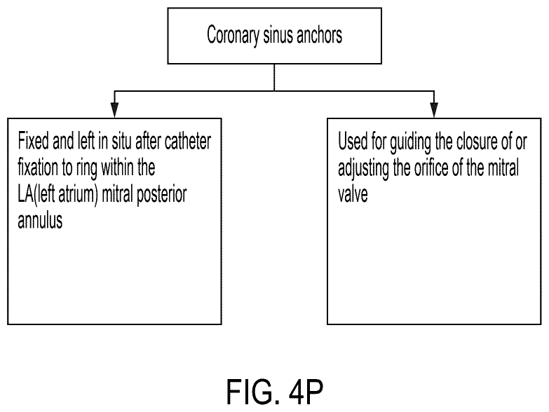

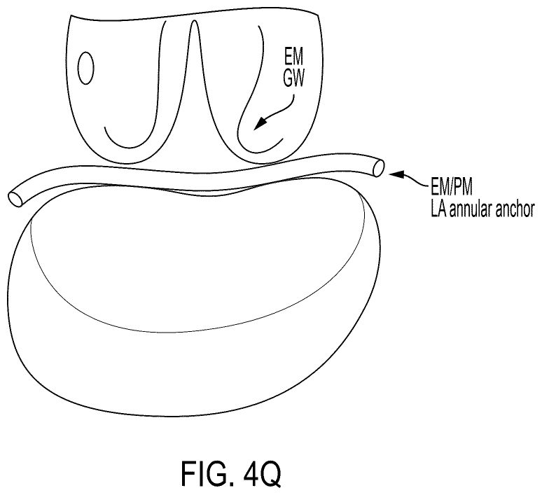

With reference now to FIGS. 3A-4Q, various functional views are shows according to various embodiment.

With reference to FIGS. 3A and 3B according to one embodiment, a stent-like structure sit on the annulus of the mitral valve at the atrioventricular plane, connected to the two papillary muscles. Fixation causes the papillary muscles to move clockwise and counterclockwise. In one embodiment movement is between 2 and 12 degrees clockwise and counterclockwise, and in another embodiment movement is between 4 and 10 degrees clockwise and counterclockwise. The stent structure similar to a vascular stent structure drapes over the mitral valve annulus. With reference to FIG. 3C according to one embodiment, the two papillary muscles that the device wants to move in rotation are shown (rotation shown in the upper hand corner, and on the left hand side near the mitral valve is the mitral annular plane). The device can be inserted through the aortic valve, advanced down from the valve then back into the into the mitral valve for a temporary application. Fixation elements can grab both the papillary muscles and open like a stent, allowing for application of pulling and twisting motions at the same time or separately. This allows augmenting movement of the atrioventricular plane as well as the twisting of the two papillary muscles, both being movements that when augmented significantly improve heart function. With reference to FIGS. 3D and 3E according to one embodiment, a drive shaft is shown according to one embodiment. A sheath is advanced through the aortic valve and the device grabs or fixates to the papillary muscles. A separate component or device can deploy over the mitral valve annulus, accomplishing both the movement of the atrioventricular plane as well as the twisting of the two papillary muscles.

With reference to FIG. 3F according to one embodiment, an alternate approach is shown going from a vein going through the interatrial septum, then fixating to or grabbing the mitral annulus and the two papillary muscles. This is a transseptal-transfemoral-venous approach via the atrial septum. The biatrial system can be used if one needs to manipulate AV plane within the right side. Shown are the right upper chamber, left upper chamber and the mitral valve. The device can be advanced from the interatrial septum then deployed on the mitral valve, then a secondary device is deployed on the papillary muscles via a transseptal approach from the venous side of the heart. The combination of rotational and planar movement is shown. The rotational or twisting motion helps empty the heart more efficiently. Both the atrioventricular plane movement and papillary muscle rotational movement is impaired in people with heart failure, so providing both modes of augmentation significantly improves heart function during heart failure. With reference to FIG. 3G according to one embodiment, mitral and papillary anchoring positions are shown. With reference to FIG. 3H according to one embodiment, force per centimeter squared on the heart anatomy can be determined. Embodiments include movement of the two upper chambers as well as movement of the septum of the upper chamber. This is in addition to achieving the AV plane movement to aid cardiac output by moving the atrial septum

With reference to FIGS. 31 and 3J, the first diagram illustrates how anchors can sit. The anchor goes around the two papillary muscles. The anchoring mechanism can be similar to embodiments above that suction tissue then deploy anchors. Alternatively, where the two papillary muscles are sitting, the anchors may simply hold the bottom of them move them around as needed, fixated for example via barbs. In one embodiment, fixation feet come out and grab the muscle, deploying in a shape similar to a diamond holder. Vascular approaches may include for example transaortic, transseptal, femoral or direct access as well. An example of an apical anchor configuration is shown. With reference to FIG. 3K, deployment and fixation around the papillary muscles is shown.

With reference now to FIG. 3L according to one embodiment, shown on the left side is the SVC superior vena cava and on the interior side is the IVC inferior vena cava. The heart is oriented sideways and the mitral valve is shown. In this transcatheter approach, the device is inserted through the interatrial septum and then open into a fan shape. With reference to FIG. 3M according to one embodiment, the right atrium is on the right side and the left atrium is on the left side. The approach was advanced through the interatrial septum (the wall between the two atrium) and deployed into a fan shape. One can manipulate this plane since the anchor will connect from an upwards approach. In one embodiment, plane movement is augmented by anchoring in the left atrium above the mitral annulus. In one embodiment, anchoring includes connection to the left arterial wall. In one embodiment, anchoring includes connection along the mitral annulus. FIG. 3N shows an alternate view of the plane that will move up and down, showing how the device can wrap around the annulus. The construct is introduced across the interatrial septum and then rests on the mitral annulus. It's important that the valve is not damaged during attachment, so attachment can include a combination for fixation to the annulus and the wall as well for better reinforcement and to avoid damage the valve. CS—coronary sinus is depicted, again showing how the construct is introduced across the interatrial septum and then rests on the mitral annulus.

With reference to FIG. 3O according to one embodiment, there are structures behind the coronary sinus which is the vein that goes behind the valve of the heart (CS: coronary sinus; AML: anterior mitral leaflet; PML: posterior mitral leaflet). A magnetic guide wire can be placed in these structures to provide a fiducial point and provide fixation. With reference to FIG. 3P according to one embodiment, a coronary sinus opening is shown (showing a wire and a construct introduced via the coronary sinus opening around the mitral annulus; IAS: inter atrial septum). A surgeon can advance the guide wire through this opening. The marker is outside of the valve in that van and provides a fiducial marker. Another marker can be placed near the interatrial septum, which is basically the wall between the two atria, the figure showing the demarcation. Similarly, FIG. 3Q shows an approach going through the interatrial septum, one marker advanced along the back wall and another along the front wall. The back wall of the valve and the coronary sinus is shown, marking the coronary. Another marker is positioned in the aortic valve, so both a posterior and anterior marker are provided. This shows how the construct would be introduced via the SVC and goes across the atrial septum to go around the mitral plane and the relations of various valves. FIG. 3R essentially shows the same thing once you have entered into the left atrium. The septum is the wall between the right atrium and the left atrium. Once you enter it, distances are very short (e.g. 2-4 centimeters), so the distances are very short for going around the annulus. The coronary sinus is just behind it, and the diagram illustrates the transseptal view. If a prediction marker is placed in the valve now which is separate, fiducial markers and points for anchoring prior to deployment are easily provided.

With reference now to FIG. 3S according to one embodiment, a partial view inside the coronary sinus is shown. With a magnetic core inside the left atrium, attachment and fixation are provided. One component is inside, one is outside, and because of a change to negative polarity the components are attracted. As shown previously in FIG. 1D, a tissue plane between magnetic components allows for anatomical fixation. Sliding magnet spheres bunch up tissue by pulling on it, moving along the plane. Electromagnets can be utilized and powered on when components are at the target location.

With reference now to FIG. 3T according to one embodiment, the fan can open upon deployment. Once the fan opens up, a back-and-forth motion can be actuated, and the fan can also be sized smaller and bigger. 20% to 25% of heart output comes from this this motion. An alternative embodiment is shown in FIG. 3U, showing more of a clam-shaped geometry utilizing thin filaments.

With reference to FIG. 3V according to one embodiment, sample dimensions for the delivery sheath are shown. 18 to 24 French will be utilized in one embodiment, however a small size for example 9

With reference to FIG. 3W according to one embodiment, an anchoring configuration is shown with a device having a flexible and non-flexible portion. The flexible can bunch up the tissue needed for anchoring. With reference to FIGS. 3X and 3Y according to one embodiment, alternatives are shown such as preformed Nitinol which can be in linear form and collapsable for advancement within the sheath then deployed and taking the preformed shape. Flexibility in certain sections allows the tissue to bunch along a particular portion.

With reference to FIG. 3Z according to one embodiment, the relationship of the aortic valve and the anterior leaflet of the mitral valve is shown. A guide wire can be inserted into each side for providing fiduciary points and fixation points for attaching components. Depicted are a NCC: non coronary cusp of aortic valve, and AML: anterior mitral leaflet. With reference to FIGS. 4A and 4B, the aorta is shown with the left coronary opening and the right coronary opening. Fixation points are shown for placement of electromagnets. Counteracting forces inside and outside the left upper chamber can hold components in place. One component is within the aorta and other within the left atrium for fixation. With reference to FIGS. 4C-4E alternative functional embodiments are shown. FIG. 4C depicts the next evolved step if the patient has mitral disease and needs both the new valve and AV plane movement. FIG. 4D shows counter magnetic fixation, the below is the coronary sinus shown in open view. FIG. 4E shows how angular motion can be affected with multiplication of force by using pully and lever system within the construct (nitinol or similar material). In one embodiment, a bucket handle motion can be implemented to provide linear motion. The motion goes back and forth with the cam and pully system. The device can be activated to match detected heart rhythm. The device only needs to be activated for depression. With reference to FIG. 4F according to one embodiment, when the atrioventricular plane device is deployed, an electrical cable similar to a pacing cable can be placed and a pocket under the clavicle under the collar bone is utilized for positioning the controller and the battery. Depicted are AVPD device via atrial septum (right), controller and battery (left), and venous (top) showing brachiocephalic vein entry like a pacemaker lead. With reference to FIG. 4G according to one embodiment, the anatomy from behind is shown in an alternate view.

With reference now to FIG. 4H according to several embodiments, there are multiple ways to apply anchors. One embodiment utilizes screws, and one embodiment utilizes an arrow with a reverse opening. One embodiment utilizes suction, which sucks the tissue then deploys the anchors. When the tissue is flat surgeons are trying to put in a screw, it often doesn't have much strength. However, by bunching the tissue up then going across the tissue, a much bigger surface area of tissue is available to grab. Other attachment mechanisms may include for example cryo, such as liquid nitrogen, liquid CO2, a cryoprobe, etc. to hold once the fiducial markers confirm proper placement. With reference now to FIGS. 4I-4K according to several embodiments, the diagram is showing an example of how a tissue anchor can be implemented. The tissue is being suctioned inside the tunnel and once it is suctioned inside, the surgeon can deploy an anchor. This way the anchor can be deployed into a thick cross-section of tissue instead of a flat screwing. The anchors can deploy via a push and pull mechanism, deploying about 4 mm upon being released from the sheath. As shown specifically in FIG. 4K, clips can also go over the ring for added stability. A second device can telescope over the initial device for providing clips on top. The clips can be in addition to the anchors underneath.

With reference to FIG. 4L according to one embodiment, a multi-piece ring can include a gear and cam system. So if you want to move just one part, that single part can be moved while the remainder stays fixed. With reference to FIG. 4M according to one embodiment, a linear actuator and tensor system is illustrated. The patient's indication will help decide what system to choose. If it is acute then more multiple moving parts may be more appropriate. However if it is chronic, less moving part may be preferable since less parts typically lead to a more durable device. With reference to FIGS. 4N and 4O according to one embodiment, additional embodiments for a construct for bunching tissue are shown. With reference to FIG. 4P according to one embodiment, an aortic valve is shown. These are the two anchors electromagnetic anchors for the the anterior wall of the mitral valve. The anterior wall needs to be supported with this structure. The posterior wall is supported by the coronary sinus.

### Experimental Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

The experimental example utilizes a device and method for improving heart function in an animal model. The experimental example is setup to see how a device focused on improving atrioventricular plane displacement can impact heart function during heart failure.

Animal Experimental Data Parameters:
Porcine preparation (n=12)
Acute Myocardial infarction by ligating LAD and reperfusion
Baseline and post AMI measurements
Insertion of device
Augmentation of device to baseline excursion
Measurements with augmentation of 7 mm of AVPD

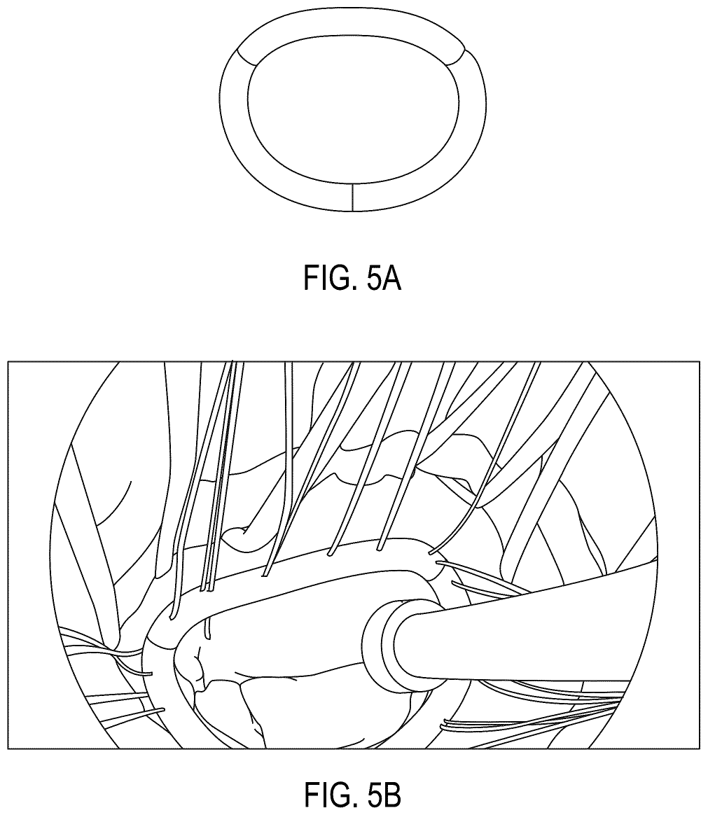

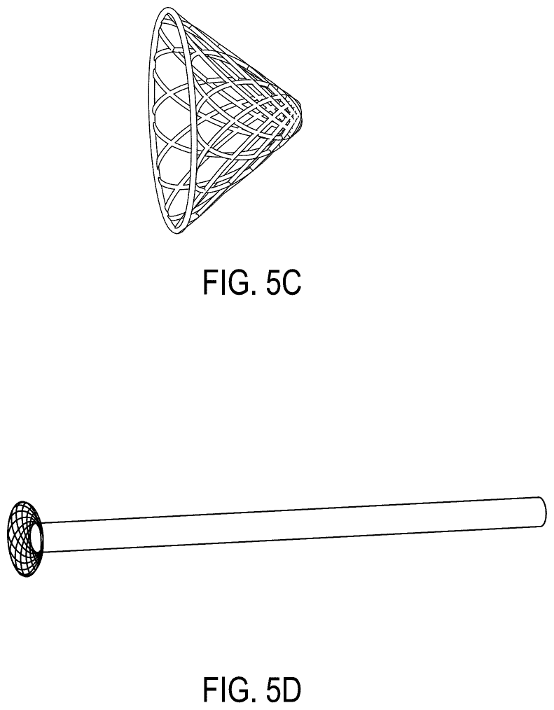

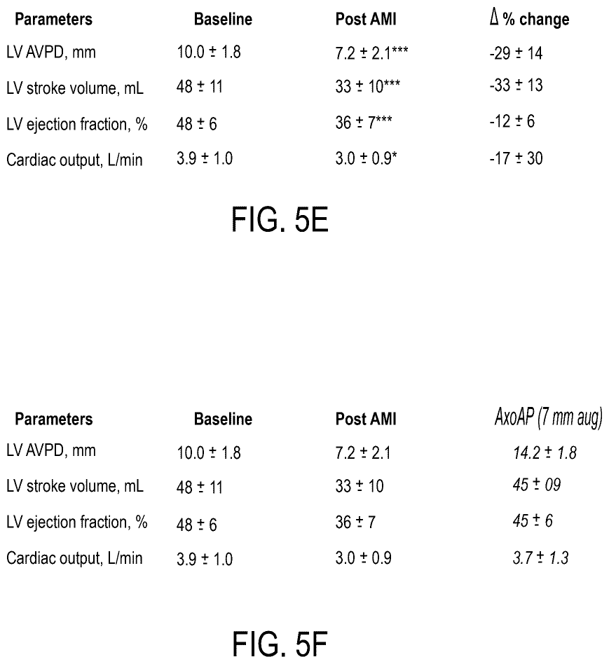

While the animals were experiencing a heart attack, the atrioventricular plane was moved back and forth to improve heart output. Atrioventricular plane displacement was augmented by up to 7 mm. The device utilized an annuloplasty ring, which is a ring typically used for tightening valves (FIG. 5A). Annuloplasty rings are typically fibrous and made of biocompatible materials such as titanium or polymers and come in various sizes to accommodate different anatomies. Structurally, annuloplasty rings are often circular or semi-circular in shape, with an adjustable diameter to fit snugly around the valve annulus. They may also feature flexible or rigid components, depending on the specific surgical approach and patient needs. The annuloplasty ring was sutured along the atrioventricular plane (e.g. at the mitral valve annulus) and connected to a linear actuator to move the atrioventricular plane back and forth by 7 mm (FIG. 5B). An alternate embodiment as shown in FIGS. 5C and 5D utilizes a webbed conical structure connected to an actuator disposed along the axis perpendicular to the atrioventricular plane for producing a back-and-forth movement by 7 mm.

With reference now to the experimental results in FIG. 5E, baseline LV atrioventricular plane displacement was about 10 mm, then during the animal heart attack it reduced to about 7.2 mm (post AMI) which reflects about 30% percent change. Stroke volume, ejection fraction and cardiac output also reduced. Referencing now FIG. 5F, the device was able to augment the heart with about 7 mm of atrioventricular plane displacement improvement, increasing movement to about 14.2 mm. This returned stroke volume and ejection fraction almost back to baseline. Heat function was restored from the criteria of a failed or struggling hard, returning function close to baseline.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

### REFERENCES

[1] Shoja M M, Agutter P S, Loukas M, Benninger B, Shokouhi G, Namdar H, et al. Leonardo da Vinci's studies of the heart. Int J Cardiol 2013; 167:1126-33.
[2] Bowman A W, Kovacs S J. Assessment and consequences of the constant-volume attribute of the four-chambered heart. Am J Physiol Heart Circ Physiol 2003; 285: H2027-33.
[3] Carlsson M, Ugander M, Mosen H, Buhre T, Arheden H. Atrioventricular plane displacement is the major contributor to left ventricular pumping in healthy adults, athletes, and patients with dilated cardiomyopathy. Am J Physiol Heart Circ Physiol 2007; 292: H1452-9.
[4] Hall J, Guyton A. Textbook of medical physiology. 12th ed. Philadelphia, USA: Saunders Elsevier; 2011.
[5] Kocica M J, Corno A F, Carreras-Costa F, Ballester-Rodes M, Moghbel M C, Cueva C N C, et al. The helical ventricular myocardial band: global, three-dimensional, functional architecture of the ventricular myocardium. Eur J Cardiothorac Surg 2006; 29 (Suppl. 1): S21-40.
[6] Watkins M W, LeWinter M M. Physiologic role of the normal pericardium. Annu Rev Med 1993; 44:171-80.
[7] Fritz T, Wieners C, Seemann G, Steen H, Dössel O. Simulation of the contraction of the ventricles in a human heart model including atria and pericardium: finite element analysis of a frictionless contact problem. Biomech Model Mechanobiol 2014; 13:627-41.
[8] Hamilton W F, Rompf J H. Movements of the base of the ventricle and the relative constancy of the cardiac volume. Am J Physiol—Leg Content 1932; 102:559-65.
[9] Hoffman E A, Ritman E L. Invariant total heart volume in the intact thorax. Am J Physiol 1985; 249: H883-90.
[10] Carlsson M, Cain P, Holmqvist C, Stahlberg F, Lundback S, Arheden H. Total heart volume variation throughout the cardiac cycle in humans. Am J Physiol Heart Circ Physiol 2004; 287: H243-50.
[11] Lundbäck S. Cardiac pumping and function of the ventricular septum. Acta Physiol Scand Suppl 1986; 550:1-101.
[12] Lundbäck S, Edfors A, Johnsson J. Computer based system adapted to create a representation of the pumping action of a heart. U.S. Pat. No. 7,239,987 B2; 2007.
[13] Lundbäck S. Heart Cluster State Machine Simulating the Heart. U.S. 20080154142 A1; 2008.
[14] Steding-Ehrenborg K, Carlsson M, Stephensen S, Arheden H. Atrial aspiration from pulmonary and caval veins is caused by ventricular contraction and secures 70% of the total stroke volume independent of resting heart rate and heart size. Clin Physiol Funct Imaging 2013; 33:233-40.
[15] Rushmer R F. Initial ventricular impulse. A potential key to cardiac evaluation. Circulation 1964; 29:268-83.
[16] Hu K, Liu D, Herrmann S, Niemann M, Gaudron P D, Voelker W, et al. Clinical implication of mitral annular plane systolic excursion for patients with cardiovascular disease. Eur Heart J Cardiovasc Imaging 2013; 14:205-12.
[17] Simonson J S, Schiller N B. Descent of the base of the left ventricle: an echocardiographic index of left ventricular function. J Am Soc Echocardiogr 1989; 2:25-35.
[18] Matos J, Kronzon I, Panagopoulos G, Perk G. Mitral annular plane systolic excursion as a surrogate for left ventricular ejection fraction. J Am Soc Echocardiogr 2012; 25:969-7

What is claimed is:

1. A physiological augmentation device for improving heart function comprising:
an actuator configured to augment displacement of an atrioventricular plane of a heart.

2. The device of claim 1, wherein the displacement comprises movement along an axis substantially perpendicular to the atrioventricular plane.

3. The device of claim 1, wherein the actuator is configured to be coupled to the heart.

4. The device of claim 1, wherein the actuator is configured to be coupled to a top portion of the heart.

5. The device of claim 1, wherein the actuator comprises a fibrous ring configured to be coupled to the heart.

6. The device of claim 1, wherein the actuator is configured to be disposed substantially along the axis.

7. The device of claim 1, wherein the actuator is configured to augment displacement of the atrioventricular plane between 1 millimeter and 7 millimeters.

8. The device of claim 1, wherein the actuator is configured to augment displacement of the atrioventricular plane between 3 millimeters and 7 millimeters.

9. The device of claim 1, wherein the actuator is configured to augment displacement of the atrioventricular plane between 5 millimeters and 7 millimeters.

10. The device of claim 1, wherein the actuator is configured to augment displacement of the atrioventricular plane by about 7 millimeters.

11. The device of claim 1, wherein the actuator comprises a plurality of magnets.

12. The device of claim 11, wherein the plurality of magnets comprises a first magnet construct and a second opposing magnet construct configured to convert magnetic energy into linear motion along the axis.

13. The device of claim 12, wherein the first magnet construct is configured to energize based on a measured electric signal from the heart.

14. The device of claim 12, wherein the first magnet construct comprises at least one sliding magnet.

15. The device of claim 1, wherein the actuator comprises a rotary device configured to convert rotary motion into linear motion along the axis.

16. The device of claim 1 further comprising:
a second actuator configured to change a ring geometry within the atrioventricular plane.

17. The device of claim 1 further comprising:
a secondary element configured to augment movement of a papillary muscle for generating rotational movement.

18. A method for improving heart function comprising:
augmenting displacement of an atrioventricular plane of a heart.

19. The method of claim 18, wherein the displacement comprises movement along an axis substantially perpendicular to the atrioventricular plane.

20. The method of claim 18, wherein the displacement is initiated by an actuator.

21. The method of claim 20, wherein the actuator is coupled to the heart.

22. The method of claim 20, wherein actuator is coupled to a top portion of the heart.

23. The method of claim 20, wherein the actuator is coupled to the heart via a fibrous ring.

24. The method of claim 20, wherein the actuator is disposed substantially along the axis.

25. The method of claim 18 further comprising:
augmenting displacement of the atrioventricular plane between 1 millimeter and 7 millimeters.

26. The method of claim 18 further comprising:
augmenting displacement of the atrioventricular plane between 3 millimeters and 7 millimeters.

27. The method of claim 18 further comprising:
augmenting displacement of the atrioventricular plane between 5 millimeters and 7 millimeters.

28. The method of claim 18 further comprising:
augmenting displacement of the atrioventricular plane by about 7 millimeters.

29. The method of claim 18 further comprising:
changing a ring geometry within the atrioventricular plane.

30. The method of claim 18 further comprising:
augmenting movement of a papillary muscle for generating rotational movement.

31. The method of claim 18 further comprising:
augmenting movement of both the left and right ventricles.

32. A transcatheter method for improving heart function comprising:
advancing a catheter though a vascular system towards a heart;
deploying an actuator device from a distal opening of the catheter;
retracting the catheter through the vascular system; and
augmenting displacement of the atrioventricular plane of the heart via the deployed actuator device.

33. The transcatheter method of claim 32, wherein the displacement comprises movement along an axis substantially perpendicular to the atrioventricular plane.

34. The transcatheter method of claim 32, wherein the actuator is coupled to the heart.

35. The transcatheter method of claim 32, wherein actuator is coupled to a top portion of the heart.

36. The transcatheter method of claim 32, wherein the actuator is coupled to the heart via a fibrous ring.

37. The transcatheter method of claim 32, wherein the actuator is disposed substantially along the axis.

38. The transcatheter method of claim 32 further comprising:
augmenting displacement of the atrioventricular plane between 1 millimeter and 7 millimeters.

39. The transcatheter method of claim 32 further comprising:
augmenting displacement of the atrioventricular plane between 3 millimeters and 7 millimeters.

40. The transcatheter method of claim 32 further comprising:
augmenting displacement of the atrioventricular plane between 5 millimeters and 7 millimeters.

41. The transcatheter method of claim 32 further comprising:
augmenting displacement of the atrioventricular plane by about 7 millimeters.

42. The transcatheter method of claim 32 further comprising:
changing a ring geometry within the atrioventricular plane.

43. The transcatheter method of claim 32 further comprising:
augmenting movement of a papillary muscle for generating rotational movement.

44. The transcatheter method of claim 32 further comprising:
augmenting movement of both the left and right ventricles.
